(19) United States
(12) Patent Application Publication
(10) Pub. No.: US 2024/0218421 A1
(43) Pub. Date: Jul. 4, 2024

(54) DEVICES AND METHODS FOR DETECTING PANCREATIC LIPASE

(71) Applicant: IDEXX LABORATORIES, INC., Westbrook, ME (US)

(72) Inventors: Arvind Dev, Westbrook, ME (US); Adam St. Hilaire, Westbrook, ME (US); Thomas Jordan, Westbrook, ME (US); Paige Pomeroy, Westbrook, ME (US)

(21) Appl. No.: 18/541,764

(22) Filed: Dec. 15, 2023

Related U.S. Application Data
(60) Provisional application No. 63/435,766, filed on Dec. 28, 2022.

(51) Int. Cl. C12Q 1/44

(52) U.S. Cl. CPC C12Q 1/44

(57) ABSTRACT

Methods and devices for determining pancreatic lipase in an animal sample are provided. In one aspect, the device includes a basic buffer layer comprising one or more bile salts and calcium; an isolation layer comprising colipase; an indicator layer comprising a chromogenic substrate such as DGGR. The device may also include and a system for optically determining the amount of a development of a color change. The methods include the use of the device to detect pancreatic lipase in animal samples.

### CROSS REFERENCE TO RELATED APPLICATION

This application claims the benefit of U.S. Provisional Application No. 63/435,766, filed Dec. 28, 2022, which is incorporated by reference herein in its entirety.

### FIELD

The present disclosure relates to devices and methods for determining the presence and/or quantity of pancreatic lipase in a sample. The devices and methods may be used in the diagnosis and management of pancreatitis in animals.

### BACKGROUND

Lipases are glycoprotein triglyceride hydrolases which catalyze the cleavage of triglycerides to diglycerides with subsequent formation of monoglycerides and fatty acids. Excess serum lipase is a symptom of pancreatitis, a disease of the pancreas characterized, in part, by excess lipase, protease and amylase production. Excess lipase production may, in turn, lead to numerous symptoms ranging from mild discomfort to death, depending, in part, on the severity of the disease and the extent of lipase overproduction.

Pancreatic lipases have for many years been important clinical chemistry parameters for the differential diagnosis of diseases of the pancreas. Numerous methods, including enzymatic assays, have been described for detecting lipase. Generally, however, these assays have had poor correlation between lipase activity determined in serum and the extent of damage to the pancreas. In particular, enzymatic assays for lipase have been found to be not specific enough to distinguish lipase activity originating from organs other than the pancreas.

Colorimetric tests that are specific for pancreatic lipase in humans, which are based on the cleavage of a chromogenic lipase substrate 1,2-O-dilauryl-rac-glycero-3-glutaric acid-(6-methyl-rsorufin) ester (“DGGR”), have been described. For example, Lipase Colorimetric Assay reagents are available from Roche Diagnostics, GmbH (Mannheim Germany). This assay is sold as a kit with two separate reagents. The first reagent includes a buffer, and the DGGR substrate with a bile acid (taurodeoxy-cholate). The second reagent includes a buffer, a colipase and a cholate. The pancreatic lipase activity is determined specifically by the combination of the bile acid and the colipase used in the assay. Virtually no lipase activity is detected in the absence of the colipase. Colipase only activates pancreatic lipase, but not other lipolytic enzymes found in serum. The high amount of cholate ensures that any esterases present in the serum do not react with the chromogenic substrate due to the highly negative surface charge.

The DGGR substrate is cleaved by the catalytic action of the alkaline lipase solution to form 1,2-O-dilauryl-rac-glycerol and an unstable intermediate, glutaric acid-(6-methylresorufin) ester. This decomposes spontaneously in alkaline solution to form glutaric acid and methylresorufin. The color intensity of the red dye formed is directly proportional to the lipase activity and can be determined photometrically.

Other commercially available lipase assays include the Lipase Color Liquid assay from Sentinel Diagnostics, Milan, Italy, and the Coloripase Coloriometic Assay from Nuclin Diagnostics, Inc., Northbrook Ill.

The window for measurement of serum lipase is about 14 days after an onset of acute pancreatitis, the peak of lipase activity reached within 24 hours and decreasing after 8 to 14 days. In animals, symptoms of pancreatitis are generally non-specific, with vomiting being the most common symptom presented to the clinician. This symptom, however, is indicative of many other diseases. Currently, animal testing for pancreatic lipase is typically performed at a reference laboratory remote from the clinic, which delays the diagnosis and, therefore, the treatment of the disease. Rapid and specific clinic-based methods for pancreatic-specific lipase will allow rapid diagnosis and treatment of the patient.

### SUMMARY

One aspect of the disclosure provides a device for detecting the presence or amount of pancreatic lipase in an animal sample in which the device includes a basic buffer layer; an isolation layer that includes colipase; an indicator layer that includes a chromogenic substrate, for example 1,2-O-dilauryl-rac-glycero-3-glutaric acid-(6-methyl-resorufin) ester (DGGR); and a system for optically determining the amount of a development of a color change in the mixture of the sample, the colipase and the chromogenic substrate.

In another aspect, the disclosure provides a method for detecting the presence or amount of pancreatic lipase in an animal sample including providing a device that includes (a) a basic buffer layer; (b) an isolation layer that includes colipase; (c) an indicator layer that includes a chromogenic substrate, e.g., DGGR; and (d) a system for optically determining the amount of color change development in the mixture of at least the sample, the colipase and the chromogenic substrate; introducing the sample onto the basic buffer layer for diffusion through the basic buffer layer, the isolation layer, and the indicator layer; determining a color change in the device; and correlating the color change to the presence or amount of pancreatic lipase in the sample.

In another aspect, the disclosure provides a kit including a device as described in the various aspects and embodiments herein and instructions for use of the device.

### BRIEF DESCRIPTION OF THE DRAWINGS

The features, objects and advantages other than those set forth above will become more readily apparent when consideration is given to the detailed description below. Such detailed description makes reference to the following figures, wherein:

FIG. 1A, FIG. 1B, FIG. 1C, FIG. 1D, FIG. 1E and FIG. 1F show exemplary arrangements of layers for detecting pancreatic lipase of the disclosure.

FIG. 2A shows the percent distribution by particle diameter for emulsified DGGR particles and FIG. 2B shows a correlation function graph indicating low polydispersity by the steeply-sloped traces and near perfect superposition for the three runs.

FIG. 3A shows that the response from the analyzer using the fully-coated dry slide format shows good separation and correlation with the Spec cPL (canine) immunoassay from the Applicant. FIG. 3B shows good separation and correlation with Spec fPL (feline), immunoassays from the Applicant.

FIG. 4 shows the results of storage stability tests of dry slides of the description frozen at −20° C. for 0 days, 15 days, 1 month, 3 months, and 6 months, where no statistically significant changes in calculated concentration were observed over six months of storage.

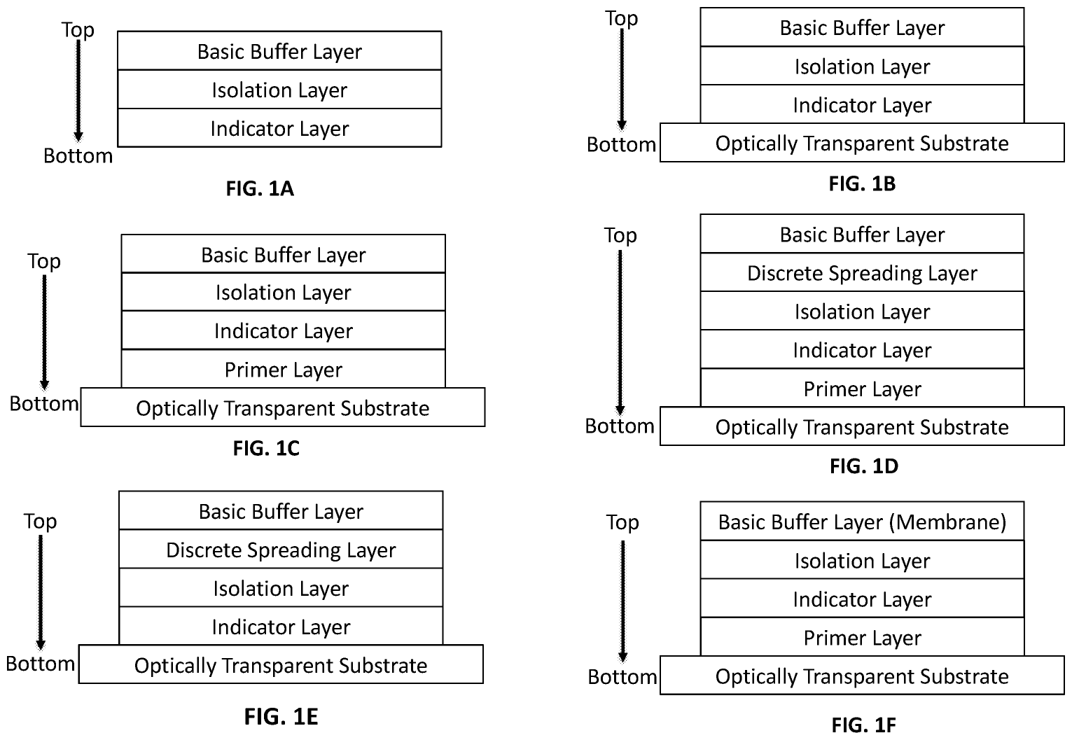

While the present methods and compositions are susceptible to various modifications and alternative forms, exemplary embodiments thereof are shown by way of example in the drawings and are herein described in detail. It should be understood, however, that the description of exemplary embodiments is not intended to limit the devices and methods to the particular forms disclosed, but, on the contrary, the intention is to cover all modifications, equivalents and alternatives falling within the spirit and scope of the devices and methods as defined by the embodiments herein and the claims below. Reference should therefore be made to the various aspects and embodiments herein and claims below for interpreting the scope of the devices and methods.

### DESCRIPTION

In one aspect, the disclosure provides devices and methods for detecting the presence or amount of pancreatic lipase in an animal sample. Embodiments of the device include a multi-layer slide that allow for flow through of the sample from a top layer to a bottom layer. A color change in a sample/reagent mixture that results from the interaction of pancreatic lipase in the sample with the reagents during the flow of the sample through the layers can be detected visually or with the appropriate equipment.

In some embodiments, the multi-layer device includes, for example, (a) a basic buffer layer; (b) an isolation layer that includes colipase; and (c) an indicator layer that includes a chromogenic substrate. The presence, amount, or rate of a color change can be detected in the indicator layer. The color may be detected visually or the device may further include or be used in a system for optically determining the amount of color or change.

Samples suitable for use in the device include a biological sample such as tissue or fluid from a human or animal including, but not limited to whole blood, plasma, and serum. Many such samples require processing, e.g., dilution, prior to analysis. Sample includes both raw samples and/or processed samples. In some embodiments, the animal sample is a canine or a feline sample.

Regarding the basic buffer layer, in some embodiments it includes a basic buffer, which in some embodiments has a pH is between about (where “about” as recited in the disclosure refers to +/−5%) pH 7 to pH 10, pH 7 to pH 9, pH 7 to pH 8, pH 8 to pH 10, pH 8 to pH 9, pH 7.5 to pH 10, pH 7.5 to pH 9.5, pH 7.5 to pH 9, pH 7.5 to pH 8.5, pH 8 to pH 10, pH 8 to pH 9.5, pH 8 to pH 9, or about any one of pH 7, pH 7.5, pH 8, pH 8.4, pH 9, pH 9.5 or pH 10. In some embodiments the basic buffer layer is dried in a buffer of between about pH 7-9.

Suitable basic buffers include, for example, MOPS (3-(N-morpholino)propanesulfonic acid), HEPES (4-(2-hydroxyethyl)-1-piperazineethanesulfonic acid), TES (N-MOBS (4-(N-Tris(hydroxymethyl)methyl-2-aminoethanesulfonic acid), Morpholino)butanesulfonic acid), DIPSO (3-(N,N-Bis[2-hydroxyethyl]amino)-2-hydroxypropanesulfonic acid), TAPSO (N-[Tris(hydroxymethyl)methyl]-3-amino-2-hydroxypropanesulfonic acid), TEA (triethanolamine), pyrophosphate (pKa₄), HEPPSO (N-(2-Hydroxyethyl)piperazine-N′-2-hydroxypropanesulphonic acid), POPSO (Piperazine-N,N′-bis(2-hydroxypropanesulfonic acid)), tricine, hydrazine, glycylglycine (pKa₂), Trizma (tris), EPPS (N-(2-Hydroxyethyl)piperazine-N′-(3-propanesulfonic acid)), HEPPS (4-(2-Hydroxyethyl)piperazine-1-propanesulfonic acid), BICINE or bicine (N,N-Bis(2-hydroxyethyl)glycine), HEPBS (4-[4-(2-Hydroxyethyl)piperazin-1-yl]butane-1-sulfonic acid), TAPS (3-{[1,3-Dihydroxy-2-(hydroxymethyl)propan-2-yl]amino} propane-1-sulfonic acid), AMPD (2-amino-2-methyl-1,3-propanediol), TABS (N-Tris(hydroxymethyl)methyl-4-aminobutanesulfonic acid), AMPSO (N-(1,1-Dimethyl-2-hydroxyethyl)-3-amino-2-hydroxypropanesulfonic acid), taurine (AES), borate (pKa₁, pKa₂, or pKa₃), CHES (2-(Cyclohexylamino)ethane-1-sulfonic acid), AMP (2-amino-2-methyl-1-propanol), glycine (pKa₂), ammonium hydroxide, CAPSO (3-(cyclohexylamino)-2-hydroxy-1-propanesulfonic acid), carbonate (pKa₂), methylamine, piperazine (pKa₂), CAPS (3-(Cyclohexylamino)-1-propanesulfonic acid), and others suitable in supporting biological reactions at basic pH levels. pKa₁ represents the negative base-10 logarithm of the first (lowest) acid dissociation constant (Ka) of a solution; pKa₂ (pKa₃, etc.) represents the negative base-10 logarithm of the second (third, etc.) acid dissociation constant (Ka) of a solution where two (three, etc.) dissociations can occur. Suitable pH buffering behavior is generally observed within +/−1 unit of a pKa value; thus, buffers with pKa values at or above 7 can be suitable for the basic buffer layer.

In particular embodiments, the basic buffer layer includes bicine buffer at pH 8.4. Generally, the amount of basic buffer substance is from 1 to 50 mg/ml, for example 5 to 45 mg/ml, 10 to 30 mg/ml or 15 to 25 mg/ml.

In some embodiments, the basic buffer layer may further include calcium that is typically provided as a calcium ion in the form of calcium salts, including, for example, calcium chloride, calcium carbonate, calcium citrate, calcium lactate, calcium sulfate, calcium malate, and the like.

In some embodiments, the basic buffer layer further comprises colipase, which colipase may be present in concentrations of from about 1 mg/L to about 50 mg/L for example 5 to 45 mg/L, 10 to 30 mg/L or 15 to 25 mg/L. In some embodiments comprising colipase in the basic buffer layer, the isolation layer does not contain colipase.

In some embodiments, the basic buffer layer further includes one or more surfactants. Suitable surfactants can include those having a carboxyl group, a sulfonic acid group, a sulfate group or a phosphate as a hydrophilic group. Anionic surfactants having a sulfonic acid group include alkylbenzenesulfonate (e.g., sodium dodecylbenzenesulfonate; SDBS), alkylnaphthalenesulfonate, alkylsulfate, a polyoxyethylene alkyl ether sulfate, α-olefin sulfonate, and N-acylmethyl taurine salts. Anionic surfactants preferably should not inhibit lipase activity or deactivate any enzyme added to devices described herein or utilized in any methods described herein.

In some embodiments, the surfactant is an alkylbenzenesulfonate with an alkyl chain containing 10 to 14 carbon atoms, and in some embodiments, sodium dodecylbenzenesulfonate is used. As a salt, a sodium salt is most common; however, potassium salts or lithium salts can also be used. Surfactants may be used in concentrations of from about 1 g/L to about 6 g/L.

In some embodiments, the one or more surfactants comprise non-ionic surfactants, such as ethoxylates, fatty alcohol ethoxylates, alkylphenol ethoxylates (APEs or APEOs), fatty acid ethoxylates, ethoxylated amines and/or fatty acid amides, terminally blocked ethoxylates, fatty acid esters of polyhydroxy compounds, fatty acid esters of glycerol, fatty acid esters of sorbitol, fatty acid esters of sucrose, alkyl polyglucosides, and the like. In some embodiments, the non-ionic surfactants according to the disclosure possess hydrophilic-lipophilic balance (HLB) in the range between about 15 and 18, HLB is a measure of its degree of hydrophilicity or lipophilicity, determined by calculating percentages of molecular weights for the hydrophilic and lipophilic portions of the surfactant molecule according to the formula HLB=20*Mₕ/M where Mₕ is the molecular mass of the hydrophilic portion of the molecule, and M is the molecular mass of the whole molecule, giving a result on a scale of 0 to 20. An HLB value of 0 corresponds to a completely lipophilic/hydrophobic molecule, and a value of 20 corresponds to a completely hydrophilic/lipophobic molecule. In some embodiments, the non-ionic surfactant is a Tergitol™, e.g., Tergitol™ 15S-30 (Dow Chemicals).

In some embodiments, the non-ionic surfactant is soluble in a polar solvent, which in some embodiments is an alcohol, e.g., methanol, ethanol, n-propanol, isopropanol, n-butanol, and the like.

In some embodiments, the basic buffer layer further comprises one or more components that facilitate sample spreading and absorption. In some embodiments, the one or more components include polymeric binders (e.g., polyacrylamide), viscosity modifiers (e.g., carboxymethyl cellulose), plasticizers (e.g., polyethylene glycol), biocidal components (e.g., Proclin), lubricants, defoaming agents (e.g., emulsified silicone), particles (or “pigments”) ranging from 1-100 μm in size, and combinations thereof as known in the art, e.g., U.S. Pat. Nos. 3,992,158, 4,258,001, and 4,670,381.

Relative concentrations of binders (and, optionally, viscosity modifiers, plasticizers, biocidal components, lubricants, defoaming agents, etc,) to elements that do not change form (such as melt-flow/film-form) or evaporate during the coating and drying process are defined by the pigment volume concentration (PVC). PVC is a well-known measurement in coating formulation to determine how much binder and “pigment” (where “pigment” is defined as particles, fillers, or other element unchanged during the coating and drying process) should be incorporated into the formulation to achieve a particular porosity in a coating. Typically, porous coatings have a pigment volume concentration >85%, as defined by the equation PVC=[ΣVp/(ΣVp+ΣVb)]×100% where ΣVp is the sum of the volumes of all “pigments” in the system and ΣVb is the sum of the volumes of all binders in the system. One of skill in the art knows that compatible binder/particle systems should be selected, where the properties of the binder are compatible with the surface chemistry of the “pigment” (particle, filler, etc).

Herein, PVCs ranging from about 87%-97%, inclusive, are suitable for the applications described herein for binders used in coatings that are of average strength or greater; weaker binders can support lower PVCs than strong binders. In certain embodiments, the favorable PVC is about 93%. Binder strength also impacts robustness of the coating to manufacturing stress.

In some embodiments, the basic buffer layer is in the form of a membrane. For example, a semi-permeable membrane (e.g., IPOC membrane (International Point of Car, Inc., Toronto, Canada)) is coated or impregnated with components of the basic buffer layer (basic buffer and, optionally, separately or two or more together, surfactant, colipase, calcium or bile salts), dried, and laminated or otherwise to the isolation layer. Preferred semi-permeable membranes have consistently sized pores and/or a regular porous structure that acts to facilitate sample spreading and absorption in addition to providing the buffering capacity from the basic buffer components.

Regarding the isolation layer, the colipase may be present in concentrations of from about 1 mg/L to about 50 mg/L for example 5 to 45 mg/L, 10 to 30 mg/L or 15 to 25 mg/L.

In some embodiments the isolation layer further includes a support matrix. The support matrix includes at least one hydrophilic polymer, and in some embodiments, the support matrix includes a mixture of two or more hydrophilic polymers. Hydrophilic polymers dissolve in, or are swollen by, water and can be natural, synthetic or semi-synthetic. Hydrophilic polymers of the disclosure include starches, pullulan, pullulan derivatives, cellulose, cellulose derivatives (e.g., carboxymethylated cellulose, methylated cellulose, hydroxyethylated cellulose, and hydroxypropylated cellulose), scleroglucan, elsinan, levan, alternant, dextran, agarose, gelatins (e.g., an acid-treated gelatin and a deionized gelatin), gelatin derivatives (e.g., a phthalated gelatin and a hydroxyacrylate graft gelatin), acrylamide polymers, copolymers including, e.g., acrylamide and a various vinyl monomers, vinylpyrrolidone polymers (e.g., polyvinylpyrrolidone), and acrylate polymers.

The hydrophilic polymers of the disclosure can be non-crosslinked or crosslinked by crosslinking reagents such as polycarboxylic acids, halogenated dicarboxylic acids, polycarboxylic anhydrides, aldehyde compounds, N-methylol compounds, isocyanate compounds, metaphosphoric acid salts, divinyl compounds, bis-aziridine, and the like. Crosslinking can also be unipolymeric (one polymer, whether alone or after crosslinking one part of a mixture of two or more hydrophilic polymers), dipolymeric (both polymers of a two polymer mixture are crosslinked in the presence of each other) or multi-polymeric (all polymers in a mixture of three or more polymers are crosslinked in the presence of each other, or a subset of two or more polymers in a mixture of two or more plus one polymers are crosslinked prior to mixing with the remaining polymers).

Mixtures of two or more hydrophilic polymers can have an equivalent proportion (as measured by, e.g., mass, moles, volume, or other measure) of each polymer, or an excess of one or more polymers over other(s). Ratios of one polymer to another polymer in a mixture of two polymers can be, e.g., about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, and ratios between. In mixtures of three or more polymers, each can vary on proportion to the others in a manner analogous to the two polymer mixture above. That is, one or more polymers can be in excess to any other polymers, or all polymers can have an equivalent proportion (again, e.g., by mass, moles, volume, or other measure). For example, a three polymer mixture (A:B:C) can have a ratio of 5:2:1, 1:1:1, 3:10:1, 4:9:1, 1:15:3, 20:1:7, and the like.

In some embodiments, the support matrix is a mixture of cellulose and pullulan, which can have an equivalent proportion of pullulan and cellulose, an excess of pullulan over cellulose, or an excess of cellulose over pullulan. Ratios of cellulose to pullulan can be, e.g., about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, and ratios between. In some embodiments, the cellulose to pullulan ratio is 2:1. Support matrix reagents may comprise up to 30 wt % of the final formulation.

In some embodiments, the isolation layer further includes an acidic buffer. In some embodiments the pH is between about pH 1 to pH 6, pH 1 to pH 5, pH 2 to pH 7, pH 2 to pH 6, pH 3 to pH 6, pH 4 to pH 6, pH 2 to pH 5, pH 3 to pH 5, pH 2.5 to pH 5.5, pH 2.5 to pH 5, pH 2.5 to pH 4.5, pH 3 to pH 5.5, pH 3.5 to pH 5.5, pH 3.5 to pH 5, or about any one of pH 2.5, pH 3, pH 3.5, pH 4, pH 4.5, pH 5, pH 5.5 or pH 6. In particular embodiments, the pH is between about pH 3.5 to pH 4.5, and in other particular embodiments, the pH of the indicator layer is about pH 4.

DGGR is unstable in neutral or basic pH conditions. Nevertheless, dry slides in the art traditionally place layers that have a neutral or basic pH in a layer adjacent to a DGGR-containing layer, which can lead, e.g., to diffusion at the layer interface and an increase in pH of the DGGR-containing layer. An increase in pH over time can lead to DGGR instability and generation of false positive resorufin-based color development. In the various aspects of the disclosure, DGGR may be maintained at an acidic pH of about 3.5 to about 4.5 until its reaction with pancreatic lipase from an animal sample by providing an isolation layer including an acidic buffer, between the basic buffer layer and the indicator layer (either with or without a discrete spreading layer). This isolation layer with its acidic pH buffer serves to further isolate the DGGR chromogenic substrate from the basic pH conditions of the basic buffer layer during manufacture and storage, which prevents or minimizes false positive pancreatic lipase activity. Only when an animal sample is affirmatively added to the basic buffer layer does the basic pH buffer from the basic buffer layer diffuse downward through each layer to the indicator layer, raising the pH to support pancreatic lipase, which requires a basic pH for optimal activity. In embodiments where the indicator layer is above the basic buffer layer, and the isolation layer is between, only when an animal sample is affirmatively added to the indicator layer does the acidic pH buffer from the indicator layer diffuse downward through each layer to the basic buffer layer, raising the pH to support any pancreatic lipase in the sample. DGGR may be used in concentrations of from about 0.2 g/L to about 10.0 g/L, e.g., about 0.3 g/L to about 9.0 g/L, or about 0.35 g/L to about 8 g/L, or about 0.4 g/L to about 7.5 g/L, or about 0.5 g/L to about 7 g/L, and the like.

Suitable acidic buffers include, for example, maleate (pKa₁), phosphate (pKa₁), glycine (pKa₁), citrate (pKa₁), glycylglycine (pKa₁), malate (pKa₁), formate, citrate (pKa₂), succinate (pKa₁), acetate, propionate, malate (pKa₂), pyridine, piperazine (pKa₁), cacodylate, succinate (pKa₂), MES (2-(N-morpholino)ethanesulfonic acid), citrate (pKa₃), maleate (pKa₂), bis-tris, carbonate (pKa₁), PIPES (piperazine-N,N′-bis(2-ethanesulfonic acid), ACES (N-(2-Acetamido)-2-aminoethanesulfonic acid), tartrate, and others suitable in supporting biological reactions at acidic pH levels. As suitable pH buffering behavior is generally observed within +/−1 unit of a pKa value; thus, buffers with pKa values below 7 can be suitable for the isolation layer. Generally, the amount of acidic buffer substance is from 1 to 50 mg/ml, for example 5 to 45 mg/ml, 10 to 30 mg/ml or 15 to 25 mg/ml.

In some embodiments, one or more layers of the device described in the disclosure, e.g., the isolation layer (and in some embodiments the indicator layer and basic buffer layer), includes one or more bile salts (i.e., salts of bile acids). Bile salts include, e.g., cholates, glycocholates, taurocholates, deoxycholates, taurodeoxycholates, chenodeoxycholates, glycochenodeoxycholates, taurochenodeoxycholates, and lithocholates. Sodium and potassium salts are most commonly observed, with sodium salts being the most common counterion. In some embodiments, the bile salts include sodium cholate, sodium deoxycholate, sodium taurocholate, sodium taurodeoxycholate, sodium deoxytaurocholate, or a mixture of two or more, and in some embodiments, the bile salt is sodium taurodeoxycholate. In some embodiments, the isolation layer further includes one or more bile salts. Bile salts may be used in concentrations of from about 2 g/L to about 30 g/L for example 5 to 25 g/L or 10 to 20 g/L.

In some embodiments, the isolation layer further includes one or more surfactants. Suitable surfactants can include those having a carboxyl group, a sulfonic acid group, a sulfate group or a phosphate as a hydrophilic group. Anionic surfactants having a sulfonic acid group include alkylbenzenesulfonate (e.g., sodium dodecylbenzenesulfonate; SDBS), alkylnaphthalenesulfonate, alkylsulfate, a polyoxyethylene alkyl ether sulfate, α-olefin sulfonate, and N-acylmethyl taurine salts. Anionic surfactants preferably do not inhibit lipase activity or deactivate any enzyme added to devices described herein.

In some embodiments, the surfactant is an alkylbenzenesulfonate with an alkyl chain containing 10 to 14 carbon atoms, and in some embodiments, sodium dodecylbenzenesulfonate is used. As a salt, a sodium salt is most common; however, potassium salts or lithium salts can also be used.

In some embodiments, the one or more surfactants comprise non-ionic surfactants as described above. In some embodiments, the non-ionic surfactants according to the disclosure possess hydrophilic-lipophilic balance (HLB) in the range between about 15 and 18, and in some embodiments, the non-ionic surfactant is soluble in a polar solvent, e.g., methanol, ethanol, n-propanol, isopropanol, n-butanol, and the like, though not limited to alcohols.

In some embodiments, the isolation layer further comprises a polyvinylpyrrolidone support matrix and a non-ionic surfactant with a HLB between 15 and 18 that is also soluble in an alcohol solvent.

Regarding the indicator layer, in some embodiments it further includes a support matrix. As with the isolation layer, the support matrix for the indicator layer includes at least one hydrophilic polymer. In some embodiments, the support matrix includes a single hydrophilic polymer, and in some embodiments the support matrix includes a mixture of two or more hydrophilic polymers. Hydrophilic polymers dissolve in, or are swollen by, water and can be natural, synthetic or semi-synthetic. Hydrophilic polymers of the disclosure include starches, pullulan, pullulan derivatives, cellulose, cellulose derivatives (e.g., carboxymethylated cellulose, methylated cellulose, hydroxyethylated cellulose, and hydroxypropylated cellulose), scleroglucan, elsinan, levan, alternant, dextran, agarose, gelatins (e.g., an acid-treated gelatin and a deionized gelatin), gelatin derivatives (e.g., a phthalated gelatin and a hydroxyacrylate graft gelatin), acrylamide polymers, copolymers including, e.g., acrylamide and a various vinyl monomers, vinylpyrrolidone polymers (e.g., polyvinylpyrrolidone), and acrylate polymers.

The hydrophilic polymers of the disclosure can be non-crosslinked or crosslinked by crosslinking reagents such as polycarboxylic acids, halogenated dicarboxylic acids, polycarboxylic anhydrides, aldehyde compounds, N-methylol compounds, isocyanate compounds, metaphosphoric acid salts, divinyl compounds, bis-aziridine, and the like. Crosslinking can also be unipolymeric (one polymer, whether alone or after crosslinking one part of a mixture of two or more hydrophilic polymers), dipolymeric (both polymers of a two polymer mixture are crosslinked in the presence of each other) or multi-polymeric (all polymers in a mixture of three or more polymers are crosslinked in the presence of each other, or a subset of two or more polymers in a mixture of two or more plus one polymers are crosslinked prior to mixing with the remaining polymers).

Mixtures of two or more hydrophilic polymers can have an equivalent proportion (as measured by, e.g., mass, moles, volume, or other measure) of each polymer, or an excess of one or more polymers over other(s). When a mixture of two polymers is present, ratios of one polymer to another polymer in a mixture of two polymers can be, e.g., about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, and ratios between. In mixtures of three or more polymers, each can vary on proportion to the others in a manner analogous to the two polymer mixture above. That is, one or more polymers can be in excess to any other polymers, or all polymers can have an equivalent proportion (again, e.g., by mass, moles, volume, or other measure). For example, a three polymer mixture (A:B:C) can have a ratio of 5:2:1, 1:1:1, 3:10:1, 4:9:1, 1:15:3, 20:1:7, and the like.

In some embodiments, the support matrix is a mixture of cellulose and pullulan, which can have an equivalent proportion of pullulan and cellulose, an excess of pullulan over cellulose, or an excess of cellulose over pullulan. Ratios of cellulose to pullulan can be, e.g., about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, and ratios between. In some embodiments, the cellulose to pullulan ratio is 2:1.

In some embodiments, the indicator layer further includes an acidic buffer. In some embodiments the pH is between about pH 1 to pH 6, pH 1 to pH 5, pH 2 to pH 7, pH 2 to pH 6, pH 3 to pH 6, pH 4 to pH 6, pH 2 to pH 5, pH 3 to pH 5, pH 2.5 to pH 5.5, pH 2.5 to pH 5, pH 2.5 to pH 4.5, pH 3 to pH 5.5, pH 3.5 to pH 5.5, pH 3.5 to pH 5, or about any one of pH 2.5, pH 3, pH 3.5, pH 4, pH 4.5, pH 5, pH 5.5 or pH 6. In particular embodiments, the pH is between about pH 3.5 to pH 4.5, and in other particular embodiments, the pH of the indicator layer is about pH 4.

Suitable buffers include, for example, maleate (pKa₁), phosphate (pKa₁), glycine (pKa₁), citrate (pKa₁), glycylglycine (pKa₁), malate (pKa₁), formate, citrate (pKa₂), succinate (pKa₁), acetate, propionate, malate (pKa₂), pyridine, piperazine (pKa₁), cacodylate, succinate (pKa₂), MES (2-(N-morpholino)ethanesulfonic acid), phosphate (pKa₁), citrate (pKa₃), maleate (pKa₂), tartrate, and others suitable in supporting biological reactions at acidic pH levels. As suitable pH buffering behavior is generally observed within +/−1 unit of a pKa value; thus, buffers with pKa values below 7 can be suitable for the indicator layer.

In some embodiments, the indicator layer further includes one or more bile salts. As described herein, bile salts include, e.g., cholates, taurocholates, deoxycholates, among others. Sodium and potassium salts are most commonly used, with sodium salts being the most common counterion. In some embodiments, the bile salts include sodium cholate, sodium deoxycholate, sodium taurocholate, sodium taurodeoxycholate, sodium deoxytaurocholate, or a mixture of two or more, and in some embodiments, the bile salt is sodium taurodeoxycholate.

In some embodiments, the indicator layer further includes one or more surfactants. Suitable surfactants can include those having a carboxyl group, a sulfonic acid group, a sulfate group or a phosphate as a hydrophilic group. Anionic surfactants having a sulfonic acid group include alkylbenzenesulfonate (e.g., sodium dodecylbenzenesulfonate; SDBS), alkylnaphthalenesulfonate, alkylsulfate, a polyoxyethylene alkyl ether sulfate, α-olefin sulfonate, and N-acylmethyl taurine salts. Anionic surfactants preferably do not inhibit lipase activity or deactivate any enzyme added to devices described herein.

In some embodiments, the surfactant is an alkylbenzenesulfonate with an alkyl chain containing 10 to 14 carbon atoms, and in some embodiments, sodium dodecylbenzenesulfonate is used. As a salt, a sodium salt is most common; however, potassium salts or lithium salts can also be used.

In some embodiments, the one or more surfactants comprise non-ionic surfactants as described above. In some embodiments, the non-ionic surfactants according to the disclosure possess hydrophilic-lipophilic balance (HLB) in the range between about 15 and 18, and in some embodiments, the non-ionic surfactant is soluble in a polar solvent, e.g., methanol, ethanol, n-propanol, isopropanol, n-butanol, and the like, though not limited to alcohols. In some embodiments, the non-ionic surfactant is a Tergitol™, e.g., Tergitol™ 15S-30.

In some embodiments, the isolation layer further comprises a polyvinylpyrrolidone support matrix and a non-ionic surfactant with a HLB between 15 and 18 that is also soluble in an alcohol solvent.

In some embodiments, the chromogenic substrate is DGGR, which in some embodiments is emulsified with a bile (acid) salt, such as a cholate, in a buffer. The bile salt is, for example, an alkali metal salt of cholic acid, taurocholic acid, deoxycholic acid, taurodeoxycholic acid, glyco-deoxycholic acid. DGGR is unstable in neutral or basic pH conditions, so DGGR may be maintained at an acidic pH of about 3.5 to about 4.5. In some embodiments, the buffer is a tartrate buffer at pH 4.0.

In some embodiments in which the chromogenic substrate is DGGR, the DGGR is not emulsified but rather a soluble or miscible component in an organic solvent or solvent system, combined with one or more hydrophilic polymers as described herein (e.g., polyvinylpyrrolidone (PVP)) that is compatible with the solvent or solvent system. Suitable solvents and solvent systems are polar organic solvents, e.g., ethanol, isopropyl alcohol, methanol and the like.

In some embodiments, the device also includes a support layer that provides a rigid or semi-rigid physical foundation for the layers of the device. In some embodiments, the support layer is optically transparent and impermeable to aqueous liquids, examples of which include film- or sheet-type transparent supports having a thickness of, e.g., 50 μm to 5 mm (and in some embodiments between about 120 μm to 180 μm) and comprising materials, in some embodiments, polymers such as polyethylene terephthalate (PET), polycarbonate of bisphenol A, polystyrene, cellulose ester (e.g., cellulose diacetate, cellulose triacetate, or cellulose acetate propionate), or the like, or other suitable materials such as glass. In some embodiments, the support layer is PET.

In some embodiments, the device also includes a primer layer on the support layer that facilitates and/or enhances the binding of the other layers of the device to the support layer. In some embodiments, the primer layer is located between the support layer and the indicator layer; whereas, in other embodiments the location of certain layers is altered, the primer layer is located between the support layer and the basic buffer layer. Alternatively, or in addition, physical or chemical activation treatment can be carried out on the support layer surface such to enhance adhesivity.

In some embodiments, the primer layer includes a hydrogel, which is a crosslinked, three-dimensional hydrophilic network that has the ability to swell but resist dissolving when placed in water or other biological fluid, such as D4 hydrogel. In some embodiments, the primer layer also includes a support matrix like cellulose and/or cellulose derivatives (e.g., carboxymethylated cellulose, methylated cellulose, hydroxyethylated cellulose, and hydroxypropylated cellulose). IN certain embodiments, the primer layer includes D4 hydrogel and cellulose.

In another aspect, the disclosure provides a device that also includes a separate, discrete, coated spreading layer, which, in some embodiments, is located between the basic buffer layer and the isolation layer.

Regarding embodiments that include a discrete spreading layer, the basic buffer layer can further include a support matrix. As described for the isolation layer and the indicator layer, the support matrix present in the basic buffer layer in some embodiments that include a discrete spreading layer includes at least one hydrophilic polymer, and in some embodiments, the support matrix includes a mixture of two or more hydrophilic polymers. Hydrophilic polymers dissolve in, or are swollen by, water and can be natural, synthetic or semi-synthetic. Hydrophilic polymers of the disclosure include starches, pullulan, pullulan derivatives, cellulose, cellulose derivatives (e.g., carboxymethylated cellulose, methylated cellulose, hydroxyethylated cellulose, and hydroxypropylated cellulose), scleroglucan, elsinan, levan, alternant, dextran, agarose, gelatins (e.g., an acid-treated gelatin and a deionized gelatin), gelatin derivatives (e.g., a phthalated gelatin and a hydroxyacrylate graft gelatin), acrylamide polymers, copolymers including, e.g., acrylamide and a various vinyl monomers, vinylpyrrolidone polymers (e.g., polyvinylpyrrolidone), and acrylate polymers.

The hydrophilic polymers of the disclosure can be non-crosslinked or crosslinked as described elsewhere herein.

Mixtures of two or more hydrophilic polymers can have an equivalent proportions or unequal proportions as described elsewhere herein. In some embodiments of this aspect of the disclosure, the support matrix included in the basic buffer layer is a mixture of cellulose and pullulan, which can have an equivalent proportion of pullulan and cellulose, an excess of pullulan over cellulose, or an excess of cellulose over pullulan. Ratios of cellulose to pullulan can be, e.g., about 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, and ratios between. In some embodiments, the cellulose to pullulan ratio is 2:1.

In some embodiments of this aspect of the disclosure, the spreading layer further includes one or more components that facilitate sample spreading and absorption. In some embodiments, the one or more components include polymeric binders, viscosity modifiers, plasticizers, biocidal components, lubricants, defoaming agents, particles ranging from 1-100 μm in size, and combinations thereof as known in the art, e.g., U.S. Pat. Nos. 3,992,158, 4,258,001, and 4,670,381. Relative concentrations can be defined as pigment volume concentrations (PVC), as described elsewhere herein.

In some embodiments of this aspect of the disclosure, the basic buffer layer is a pH of between about pH 7-9, e.g., with bicine buffer or other basic buffer as described herein, and further includes one or more surfactants and a pullulan-cellulose support matrix (each of the surfactant(s) and the support matrix as described above and herein), with the proviso that in some embodiments the basic buffer layer does not include carboxymethyl cellulose, or a polyethylene glycol, e.g., PEG 300. In some embodiments of this aspect with a discrete spreading layer present, the isolation layer does not include a surfactant (e.g., SDBS) or bile salts (e.g., cholate, taurodeoxycholate, and the like).

In particular embodiments of all aspects of the disclosure, the basic buffer layer includes bicine buffer, one of the one or more surfactants is SDBS, and the pullulan-cellulose support matrix comprises an excess of cellulose to pullulan

In some embodiments, one or more of the layers of the device described herein, e.g., the basic buffer layer, the isolation layer and the indicator layer, are coated and dried on an optically transparent substrate, either directly on the substrate or, in some embodiments, on a primer layer that facilitates and/or enhances the binding of the other layers of the device to the substrate. In some embodiments, each successive layer, from bottom to top of the device, is coated and dried prior to the addition of the next layer. For example, in embodiments with an optically transparent substrate and a primer layer, the primer layer is coated and dried, the indicator layer is coated onto the primer layer and dried, the isolation layer is coated onto the indicator layer and dried, and the basic buffer layer is coated on the isolation layer and dried.

In some embodiments, the basic buffer layer is between about 150 μm and 250 μm in thickness, the isolation layer is between about 25 μm and 75 μm in thickness, the indicator layer is between about 25 μm and 75 μm in thickness and the primer layer is between about 5 μm and 20 μm in thickness. In other embodiments, the basic buffer layer is about 200 μm in thickness, the isolation layer is about 50 in thickness, the indicator layer is about 45 μm in thickness and the primer layer is about 12 μm in thickness.

In some embodiments in which a discrete spreading layer is present, the basic buffer layer is between about 150 μm and 250 μm in thickness, the spreading layer is between about 100 μm and 200 μm, the isolation layer is between about 25 μm and 75 μm, the indicator layer is between about 25 μm and 75 μm and the primer layer is between about 5 μm and 20 μm. In certain embodiments, the basic buffer layer is about 200 μm in thickness, the discrete spreading layer is about 150 μm in thickness, the isolation layer is about 50 μm in thickness, the indicator layer is about 45 μm in thickness and the primer layer is about 12 μm in thickness.

In some embodiments, the device includes a set of colorimetric standards for relating a color change in a mixture comprising the animal sample, the bile salts, the colipase and the chromogenic substrate to an amount of pancreatic lipase in the sample. Colorimetric standards include a color chart comprising a plurality of colors for correlating a color change in a mixture comprising the sample, bile salts, colipase and chromogenic substrate to an amount of pancreatic lipase in the sample, where the mixture is created within the layers of the device after application of the sample to the top layer of the device. Colorimetric standards can also be generated with a standard curve, e.g., a plurality of known amounts of pancreatic lipase can be tested for their color development upon cleaving the glutaric acid-(6-methylresorufin) ester from the DGGR substrate, which ester decomposes spontaneously in alkaline solution to form glutaric acid and methylresorufin, the chromophore. Standard samples with no pancreatic lipase to high levels of canine or feline pancreatic lipase, as appropriate for the animal samples to be run, generate a standard curve for color versus pancreatic lipase concentration, from which animal sample pancreatic lipase concentrations can be determined.

In another aspect, the device for detecting the presence of amount of pancreatic lipase in an animal sample includes additional arrangements of layers. For instance, in some embodiments the device includes, in order from top to bottom, (i) an indicator layer, (ii) an isolation layer, (iii) a basic buffer layer and (iv) a system for optically determining the amount of a development of a color change in the mixture of the sample, the bile salts, the colipase and the chromogenic substrate. In some embodiments, such devices also include a primer layer beneath the basic buffer layer, and/or an optically transparent substrate if, for example, the various layers are not self-supporting or otherwise of desired rigidity.

In some embodiments of this aspect, the device also includes a discrete spreading layer located between the indicator layer and the isolation layer or between the isolation layer and the basic buffer layer. As with other aspects and embodiments described herein that include a discrete spreading layer, for embodiments of this aspect the basic buffer layer can further include a support matrix. The support matrix present in the basic buffer layer in some embodiments that include a discrete spreading layer includes at least one (crosslinked or non-crosslinked) hydrophilic polymer, and in some embodiments, the support matrix includes a mixture of two or more hydrophilic polymers as described elsewhere herein.

Mixtures of two or more hydrophilic polymers for embodiments of this aspect can have an equivalent proportions or unequal proportions in ratios as described elsewhere herein. In some embodiments of this aspect of the disclosure, the support matrix included in the basic buffer layer is a mixture of cellulose and pullulan, which in some embodiments, the cellulose to pullulan ratio is about 2:1.

In some embodiments of this aspect of the disclosure, the discrete spreading layer further includes one or more components that facilitate sample spreading and absorption. In some embodiments, the one or more components include polymeric binders, viscosity modifiers, plasticizers, biocidal components, lubricants, defoaming agents, particles ranging from 1-100 μm in size, and combinations and concentrations thereof as known in the art, e.g., U.S. Pat. Nos. 3,992,158, 4,258,001, and 4,670,381, and described elsewhere herein.

In some embodiments of this aspect of the disclosure, the basic buffer layer is dried in a buffer of between about pH 7-9, e.g., with bicine buffer or other basic buffer as described herein, and can further include one or more surfactants and a pullulan-cellulose support matrix (each of the surfactant(s) and the support matrix as described above and herein), with the proviso that in some embodiments the basic buffer layer does not include carboxymethyl cellulose, or a polyethylene glycol, e.g., PEG 300. In some embodiments of this aspect with a spreading layer present, the isolation layer does not include a surfactant (e.g., SDBS) or bile salts (e.g., cholate, taurodeoxycholate, and the like).

In another aspect, the disclosure provides methods for detecting the presence or amount of pancreatic lipase in an animal sample including (i) providing a device for detecting the presence or amount of pancreatic lipase in an animal sample including a basic buffer layer; an isolation layer that includes colipase; an indicator layer that includes a chromogenic substrate; and a system for optically determining the amount of color change in the mixture of the sample, the colipase and the chromogenic substrate; (ii) introducing the sample onto the basic buffer layer (or other top-most layer as described herein) for diffusion through the basic buffer layer, the isolation layer, and the indicator layer (i.e., subsequent layers); (iii) determining a color change in the device, and (iv) correlating the color change to the presence or amount of pancreatic lipase in the sample.

Foundationally, the methods are based upon the cleavage by the catalytic action of alkaline lipase solution of specific chromogenic lipase substrate DGGR, which can be emulsified with bile acids as described in U.S. Pat. No. 4,988,496 (which is incorporated herein by reference in its entirety), or dissolved in a polar solvent, e.g., methanol, ethanol, n-propanol, isopropanol, and the like. This substrate is specific for pancreatic lipase in the presence of colipase, which activates pancreatic lipase, but not other lipolytic enzymes found in serum. The DGGR reagent and colipase are available from Roche Diagnostics GmbH, (Mannheim, Germany).

In some method embodiments, the animal sample is a canine or a feline sample, for example, serum or blood, and in some embodiments the chromogenic substrate includes 1,2-O-dilauryl-rac-glycero-3-glutaric acid-(6-methyl-resorufin) ester (DGGR).

In some method embodiments, the basic buffer layer includes a basic buffer, which in some embodiments has a pH is between about pH 7 to pH 10, pH 7 to pH 9, pH 7 to pH 8, pH 8 to pH 10, pH 8 to pH 9, pH 7.5 to pH 10, pH 7.5 to pH 9.5, pH 7.5 to pH 9, pH 7.5 to pH 8.5, pH 8 to pH 10, pH 8 to pH 9.5, pH 8 to pH 9, or about any one of pH 7, pH 7.5, pH 8, pH 8.4, pH 9, pH 9.5 or pH 10. In some embodiments the basic buffer layer is dried in a buffer of between about pH 7-9.

Suitable basic buffers are described herein and include those buffers with pKa values at or above 7 and are amenable to biological reactions. In particular embodiments, the basic buffer layer includes bicine buffer at pH 8.4.

In some embodiments, the basic buffer layer may further include calcium that is typically provided as a calcium ion in the form of calcium salts, including, for example, calcium chloride, calcium carbonate, calcium citrate, calcium lactate, calcium sulfate, calcium malate, and the like.

In some embodiments, the basic buffer layer further comprises colipase, which colipase may be present in concentrations of from about 1 mg/L to about 50 mg/L, for example 5 to 45 mg/L, 10-30 mg/L or 15 to 25 mg/L. In some embodiments comprising colipase in the basic buffer layer, the isolation layer does not contain colipase.

In some embodiments, the basic buffer layer further includes one or more surfactants. Suitable surfactants are described in detail elsewhere herein, and in some embodiments, the surfactant is an alkylbenzenesulfonate with an alkyl chain containing 10 to 14 carbon atoms, and in some embodiments, sodium dodecylbenzenesulfonate is used, and in some embodiments, one or more non-ionic surfactants are used, some of which are soluble in polar solvents such as methanol, ethanol, isopropanol, and the like. As a salt, a sodium salt is most common; however, potassium salts or lithium salts can also be used.

In some embodiments, the basic buffer layer further comprises one or more components that facilitate sample spreading and absorption as described above and, e.g., U.S. Pat. Nos. 3,992,158, 4,258,001, and 4,670,381. Relative concentrations of binders (and, optionally, viscosity modifiers, plasticizers, biocidal components, lubricants, defoaming agents, etc,) to “pigments” can again be defined by the pigment volume concentration (PVC), and suitable PVCs herein ranging from about 87%-97%, inclusive.

In some embodiments, the basic buffer layer is in the form of a membrane. For example, a semi-permeable membrane (e.g., IPOC membrane (International Point of Car, Inc., Toronto, Canada)) is coated or impregnated with components of the basic buffer layer (basic buffer and, optionally, surfactant and/or colipase), dried, and laminated or otherwise to the isolation layer. Preferred semi-permeable membranes have consistently sized pores and/or a regular porous structure that acts to facilitate sample spreading and absorption in addition to providing the buffering capacity from the basic buffer components.

In some embodiments of the method, the basic buffer layer further includes a thickening agent including at least one hydrophilic polymer. Hydrophilic polymeric thickeners are described elsewhere herein but, in part, include cellulose, cellulose derivatives, acrylamide polymers, copolymers including, e.g., acrylamide and a various vinyl monomers, vinylpyrrolidone polymers (e.g., polyvinylpyrrolidone), and acrylate polymers. In particular embodiments, the basic buffer layer includes carboxymethyl cellulose.

In some embodiments of the methods described herein, the isolation layer further includes a support matrix. The support matrix includes at least one hydrophilic polymer, and in some embodiments, the support matrix includes a mixture of two or more hydrophilic polymers. Suitable hydrophilic polymers can be natural, synthetic or semi-synthetic, examples of which are described elsewhere herein. Hydrophilic polymers can be non-crosslinked or crosslinked.

Mixtures of two or more hydrophilic polymers can have an equivalent proportion of each polymer, or an excess of one or more polymers over other(s) as described elsewhere herein. In some embodiments, the support matrix is a mixture of cellulose and pullulan at a 2:1 ratio, and in some embodiments, the support matrix is polyvinylpyrrolidone.

In some method embodiments, the isolation layer further includes an acidic buffer of a composition and pH value or value range as described elsewhere herein. In embodiments, the pH range is between about pH 3.5 and pH 4.5 to help DGGR in the indicator remain stable. Suitable acidic buffers are described elsewhere herein.

In some embodiments, one or more layers, e.g., the basic buffer layer and/or the indicator layer, of the device utilized in the methods of the disclosure include one or more bile salts. As described herein, bile salts include, e.g., cholates, taurocholates, deoxycholates, among others. Sodium and potassium salts are most commonly used, with sodium salts being the most common counterion. In some embodiments, the bile salts include sodium cholate, sodium deoxycholate, sodium taurocholate, sodium taurodeoxycholate, sodium deoxytaurocholate, or a mixture of two or more, and in some embodiments, the bile salt is sodium taurodeoxycholate. In some method embodiments, the isolation layer further includes one or more bile salts.

In some method embodiments, the isolation layer further includes one or more surfactants. Suitable surfactants are described elsewhere herein and can include, in part, alkylbenzenesulfonates with an alkyl chain containing 10 to 14 carbon atoms (e.g., sodium dodecylbenzenesulfonate; SDBS), and in some embodiments, one or more non-ionic surfactants are used, some of which are soluble in polar solvents such as methanol, ethanol, isopropanol, and the like. Surfactants preferably do not inhibit lipase activity or deactivate any enzyme added to devices described herein.

In some method embodiments, the indicator layer further includes a support matrix. The support matrix includes at least one hydrophilic polymer, and in some embodiments, the support matrix includes a mixture of two or more hydrophilic polymers. Suitable hydrophilic polymers can be natural, synthetic or semi-synthetic, as well as non-crosslinked or crosslinked, examples of which are described elsewhere herein.

Mixtures of two or more hydrophilic polymers can have an equivalent proportion of each polymer, or an excess of one or more polymers over other(s) as described elsewhere herein. In some embodiments, the support matrix is a mixture of cellulose and pullulan at a 2:1 ratio.

In some method embodiments, the indicator layer further includes an acidic buffer of a composition and pH value or range as described elsewhere herein. In particular embodiments, the pH is between about pH 3.5 to pH 4.5, and in other particular embodiments, the pH of the indicator layer is about pH 4, and the acidic buffer is a tartrate buffer.

In some embodiments, the indicator layer further includes one or more surfactants. As for the isolation layer described above, suitable surfactants are described elsewhere herein and can include, in part, alkylbenzenesulfonates with an alkyl chain containing 10 to 14 carbon atoms (e.g., sodium dodecylbenzenesulfonate; SDBS). In some embodiments, the one or more surfactants comprise non-ionic surfactants as described above. In some embodiments, the non-ionic surfactants according to the disclosure possess hydrophilic-lipophilic balance in the range between about 15 and 18, and in some embodiments, the non-ionic surfactant is soluble in a polar solvent, e.g., methanol, ethanol, n-propanol, isopropanol, n-butanol, and the like, though not limited to alcohols. In some embodiments, the non-ionic surfactant is a Tergitol™, e.g., Tergitol™ 15S-30.

In some embodiments, the isolation layer further comprises a polyvinylpyrrolidone support matrix and a non-ionic surfactant with a HLB between 15 and 18 that is also soluble in an alcohol solvent Surfactants preferably do not inhibit lipase activity or deactivate any enzyme added to devices described herein.

In some method embodiments, the chromogenic substrate in the indicator layer is DGGR, which in some embodiments is emulsified with one or more bile (acid) salts, such as a cholate, in a buffer. Suitable bile salts are described elsewhere herein and include, e.g., alkali metal salt of cholic acid, taurocholic acid, desoxycholic acid, taurodesoxycholic acid, glyco-desoxycholic acid and the like.

In some method embodiments, the chromogenic substrate is DGGR in which the DGGR is not emulsified but rather a soluble or miscible component in an organic solvent or solvent system, combined with one or more hydrophilic polymers as described herein (e.g., a vinylpyrrolidone polymer such as polyvinylpyrrolidone) that is compatible with the solvent or solvent system. Suitable solvents and solvent systems are polar organic solvents, e.g., ethanol, isopropyl alcohol, methanol and the like.

In some method embodiments, the device also includes a support layer that provides a rigid or semi-rigid physical foundation for the layers of the device. The support layer is optically transparent and impermeable to aqueous liquids, as well as of a thickness and composition as describe elsewhere. In some embodiments, the support layer is made of PET.

In some method embodiments, the device also includes a primer layer on the support layer that facilitates and/or enhances the binding of the other layers of the device to the support layer. In some embodiments, the primer layer is located between the support layer and the indicator layer; whereas, in other embodiments the location of certain layers is altered, the primer layer is located between the support layer and the basic buffer layer. Alternatively, or in addition, physical or chemical activation treatment can be carried out on the support layer surface such to enhance adhesivity. As described more completely elsewhere herein, in some embodiments, the primer layer includes a hydrogel, such as D4 hydrogel. In some embodiments, the primer layer also includes a support matrix like cellulose and/or cellulose derivatives (e.g., carboxymethylated cellulose, methylated cellulose, hydroxyethylated cellulose, and hydroxypropylated cellulose). In certain embodiments, the primer layer includes D4 hydrogel and cellulose.

In some embodiments, the chromogenic substrate is emulsified DGGR, at least one surfactant is SDBS, at least one of the isolation layer and the indicator layer further comprises a support matrix comprising pullulan-cellulose.

In some embodiments, the chromogenic substrate is DGGR dissolved in a polar solvent, at least one surfactant is a non-ionic surfactant with a Hydrophobic-Lipophilic Balance of between about 15-18, and at least one of the isolation layer and the indicator layer further comprising a support matrix comprising PVP.

In another method aspect, the disclosure provides methods for detecting the presence or amount of pancreatic lipase in an animal sample, wherein the device further includes a discrete spreading layer. In some embodiments, the discrete spreading layer is located between the basic buffer layer and the isolation layer.

For embodiments that include a discrete spreading layer, the basic buffer layer can further include a support matrix. As described for the isolation layer and the indicator layer, in some embodiments that include a discrete spreading layer the support matrix, if present, in the basic buffer layer includes at least one hydrophilic polymer, or a mixture of two or more hydrophilic polymers, which polymers can be natural, synthetic or semi-synthetic. Hydrophilic polymers of the disclosure are described elsewhere herein but include, in part, pullulan, pullulan derivatives, cellulose, cellulose derivatives (e.g., carboxymethylated cellulose, methylated cellulose, hydroxyethylated cellulose, and hydroxypropylated cellulose), acrylamide polymers, among others. The hydrophilic polymers of the disclosure can be non-crosslinked or crosslinked as described elsewhere herein.

Mixtures of two or more hydrophilic polymers can have an equivalent proportions or unequal proportions as described elsewhere herein. In some embodiments of this aspect of the disclosure, the support matrix included in the basic buffer layer is a mixture of cellulose and pullulan, which in some embodiments, are in a cellulose to pullulan ratio of 2:1.

In some embodiments of this method aspect of the disclosure, the discrete spreading layer further includes one or more components that facilitate sample spreading and absorption. In some embodiments, the one or more components include polymeric binders, viscosity modifiers, plasticizers, biocidal components, lubricants, defoaming agents, particles ranging from 1-100 μm in size, and combinations thereof as known in the art, e.g., U.S. Pat. Nos. 3,992,158, 4,258,001, and 4,670,381.

In some embodiments, the basic buffer layer further includes one or more surfactants. Suitable surfactants are described in detail elsewhere herein, and in some embodiments, the surfactant is an alkylbenzenesulfonate with an alkyl chain containing 10 to 14 carbon atoms, and in some embodiments, sodium dodecylbenzenesulfonate is used. As a salt, a sodium salt is most common; however, potassium salts or lithium salts can also be used.

In some embodiments, the basic buffer layer further includes a polyethylene glycol (or PEG alternative) as described elsewhere herein. In particular embodiments, one or more of PEG 200 through PEG 600 are included, and in other embodiments, one or more of PEG 200 through PEG 400 are included, and in some embodiments, PEG 200, PEG 300, or PEG 400 is included, and in some embodiments, PEG 300 is included.

In some embodiments of this method aspect, the basic buffer layer is dried in a buffer of between about pH 7-9, e.g., with bicine buffer or other basic buffer as described herein, and further includes one or more surfactants and a pullulan-cellulose support matrix (each of the surfactant(s) and the support matrix as described above and herein), with the proviso that in some embodiments the basic buffer layer does not include carboxymethyl cellulose, or a polyethylene glycol, e.g., PEG 300. In some embodiments of this aspect with a discrete spreading layer present, the isolation layer does not include a surfactant (e.g., SDBS) or bile salts (e.g., cholate, taurodeoxycholate, and the like).

In particular embodiments of all aspects of the disclosure, the basic buffer layer includes bicine buffer, one of the one or more surfactants is SDBS, and the pullulan-cellulose support matrix comprises an excess of cellulose to pullulan.

In embodiments of this aspect, the basic buffer layer, the isolation layer, and the indicator layer are of a layer thickness described elsewhere herein, whether with or without a discrete spreading present.

In some embodiments of this aspect, the device includes a set of colorimetric standards for relating a color change in a mixture comprising the animal sample, the bile salts, the colipase and the chromogenic substrate to an amount of pancreatic lipase in the sample, which are described in more detail elsewhere herein.

In another aspect, the disclosure provides methods for detecting the presence or amount of pancreatic lipase in an animal sample in which the device includes alternative arrangements of layers. For instance, in some embodiments the device includes (i) an indicator layer, (ii) an isolation layer, (iii) a basic buffer layer and (iv) a system for optically determining the amount of a development of a color change in the mixture of the sample, the bile salts, the colipase and the chromogenic substrate. In some embodiments, such devices also include a primer layer beneath the basic buffer layer, and/or an optically transparent substrate if, for example, the various layers are not self-supporting or otherwise of desired rigidity.

In some embodiments of this method aspect, the device includes a discrete spreading layer located between the indicator layer and the isolation layer or between the isolation layer and the basic buffer layer. As with other aspects and embodiments described herein that include a discrete spreading layer, for embodiments of this aspect the basic buffer layer can further include a support matrix. The support matrix, if present, in the basic buffer layer in embodiments that include a discrete spreading layer includes at least one (crosslinked or non-crosslinked) hydrophilic polymer, and in some embodiments, the support matrix includes a mixture of two or more hydrophilic polymers as described elsewhere herein. Such mixtures of two or more hydrophilic polymers can have an equivalent proportions or unequal proportions in ratios as described elsewhere herein. In some embodiments of this aspect of the disclosure, the support matrix included in the basic buffer layer is a mixture of cellulose and pullulan, which in some embodiments, the cellulose to pullulan ratio is about 2:1.

In some embodiments of this aspect of the disclosure, the discrete spreading layer further includes one or more components that facilitate sample spreading and absorption. In some embodiments, the one or more components include polymeric binders, viscosity modifiers, plasticizers, biocidal components, lubricants, defoaming agents, particles ranging from 1-100 μm in size, and combinations thereof as known in the art, e.g., U.S. Pat. Nos. 3,992,158, 4,258,001, and 4,670,381.

In some embodiments of this method aspect, the basic buffer layer is dried in a buffer of between about pH 7-9, e.g., with bicine buffer or other basic buffer as described herein, and further includes one or more surfactants and a pullulan-cellulose support matrix (each of the surfactant(s) and the support matrix as described above and herein), with the proviso that in some embodiments the basic buffer layer does not include carboxymethyl cellulose, or a polyethylene glycol, e.g., PEG 300. In some embodiments of this aspect with a discrete spreading layer present, the isolation layer does not include a surfactant (e.g., SDBS) or bile salts (e.g., cholate, taurodeoxycholate, and the like).

In all aspects of the disclosure, canine or feline pancreatic lipase can be detected in blood samples, including whole blood, plasma or serum. When the sample is, e.g., a serum sample, it includes a fraction, aliquot, droplet, portion, or volume of serum, taken as whole blood from a patient source that is collected in the standard way using standard sampling tubes such as Li-, Na- or NH₄-heparin plasma. Blood samples may be centrifuged before performing the method of the invention. EDTA, oxalate, fluoride, or citrated plasma is less desirable for storing test blood samples as they inhibit lipase activity. A test sample may be taken from an animal source using techniques known to one skilled in the art, including but not limited to, those described or referred to in “Manual of Clinical Microbiology” (6th ed.) 1995, edited by P. R. Murray, E. J. Baron, M. A. Pfaller, F. C. Tenover, and R. H. Yolken.

An animal sample is applied to the top layer of the device for detecting the presence or amount of pancreatic lipase in an animal sample. Samples are typically between about 1 μL to 25 μL, or between about 2 μL to 20 μL, or about 3 μL to 15 μL, or about 4 μL and 12 μL, or about 5 μL to about 10 μL, or about 6 μL to about 8 μL, or about 5 μL to about 20 μL, or about 1 μL to about 12 μL, or about 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL, 21 μL, 22 μL, 23 μL, 24 μL, or about 25 μL.

After an animal sample is applied to the top layer of the device, color development will occur over time if there is pancreatic lipase present in the sample. Color development generally occurs over about 5-15 minutes, at which time the color change can be determined by human or machine-based (e.g., spectrophotometric) observation. Observed color change than be compared to standards as described elsewhere herein and correlated to the presence or amount of pancreatic lipase.

In yet another aspect, the disclosure provides a kit for performing the method that includes the device for detecting the presence or amount of pancreatic lipase in an animal sample and instructions for use of the device. The kit components may be enclosed in a further sealed container for shipment and storage, such as a foil pouch, and may include a desiccant. The kit may also include specimen collection tubes for serum collection such as Li-, Na- or NH₄-heparin tubes.

For all aspects of the disclosure, one or more coating techniques can be used to create the various layers of the device for detecting the presence or amount of pancreatic lipase in an animal sample. For example, knife-over-air coating (floating knife), knife-over-roll coating, roll coating, dipping, roll-to-roll coating (e.g., Dynacoat™ from Frontier, Towanda, PA), web coating, kiss coating, gravure coating, metering rod or Myer bar coating, comma direct coating, comma reverse coating, reverse roll coating, slot orifice coating, calendaring, immersion or dip coating, and curtain coating may be used for the basic buffer layer, the isolation layer, the indication layer, and, where applicable, the discrete spreading layer, and the primer layer.

For example, for an embodiment that includes an optically transparent substrate, knife-over-air coating can be used to sequentially apply each layer. The knife-over-air coating process involves a knife or blade positioned vertically above the substrate which is being supported on either side by two rollers. Coating weights using this technique are derived from the distance between the two rollers and the tension of the substrate The blade can also be raised and lowered to apply additional tension to impact the coating weight and level of penetration (assuming any significant substrate permeability), which will affect adhesion and handle. Knife-over-air coating involves the blade being positioned above the substrate, which is supported by a roller below. The blade can be raised or lowered to obtain different coating thicknesses, and the shape of the blade can be altered to complement the viscosity and rheology of the coating medium.

Layers can be coated sequentially, from bottom layer to top layer. In some embodiments, at least one layer is dried for a time sufficient to prevent or minimize mixing with the subsequently coated layer.

For all aspects of the disclosure, some embodiments can include a filtering layer that can remove unwanted particulate from the sample. For example, the filtering layer can remove red blood cells to prevent the cells from interfering with the determination of the development of a color in a reagent layer, e.g., the indicator layer.

### EXAMPLES

### Example 1—Primer Layer Preparation and Deposition

10 g/kg cellulose was added to 990 g/kg of 10 wt % HydroMed D4 solution (AdvanSource) into a mixing vessel. The mixture was agitated to disperse the cellulose. Manual shaking was sufficient for dispersal prior to rolling in a cylindrical container at about 7.25 cm/sec for at least 12 hours at room temperature. The hydrogel-cellulose mixture was then added to a Dynacoater® (Frontier) and coated onto an optically transparent substrate, in this case Melinex® (DuPont Teijin Films™). Fluid circulation during operation of the coater sufficiently maintained cellulose dispersal. In this example, the shim width was 6 inches, with a thickness of 0.007 inches. The web gap was 150 μm; the web speed was 3 feet/minute; the unwind tension was 5 lbs; and the rewind torque at 20%. The dryer profile was 110×8 (i.e., all eight dryers were set to 110 F), with a vacuum of 0.3 inches. The fluid delivery rate was 11.76 mL/minute resulting in a derived wet thickness of 84 μm and a measured dry thickness of 7.5 μm. One of ordinary skill understands that different coating techniques can be used to achieve similar results and that application parameters can be varied to tune layer characteristics, e.g., thickness.

### Example 2—Indicator Layer Preparation and Deposition

### I. DGGR High-Shear Emulsification Procedure (20-30 mL Scale)

22.75 grams of DGGR buffer (9.3 mM tartrate pH 4.0 at RT, 0.1 mM calcium chloride, 2.6 g/kg SDBS and 2.4 g/kg sodium taurodeoxycholate) was added to a tared mixing container (here a 50 ml conical vial).

The mixing container was placed in an ice bath vertically, with its position secured to avoid movement while undergoing shear treatment. The ice bath and conical was placed under the shear mixer (e.g., Silverson, Rotosolver or Scott Turbon mixers), and the mixing head was lowered so the liquid level was around the 38 mL mark (when using a conical vial).

The DGGR/1-propanol stock solution (25 g/L DGGR and balance 1-propanol) was placed on a scale and tared. 1.85 grams of DGGR/1-propanol solution was aspirated using a needle and syringe. Over the course of 1-2 minutes, while slowly increasing the mixing speed (e.g., to 6500 RPM on a Silverson mixer), the DGGR concentrate was slowly added to the DGGR buffer in the mixing container at a rate of about 2 mL/minute. The DGGR solution was mixed on the Silverson at 6500 RPM for 30 minutes.

After 30 minutes, mixing was stopped and the mixer head was raised to allow the liquid in the assembly to drain into the mixing container for 1-2 minutes to maximize recovery. The Silverson was cleaned with 70% IPA by inserting the mixing head into an empty 50 ml conical vial, adding 25-30 mL of IPA to the conical vial, submerging the mixing head and mixing at 3000 RPM for 20 seconds. The wash IPA was combined with the shear-mixed DGGR and stored in a refrigerator at 4-8° C.

### II. DGGR Emulsion Quality Control

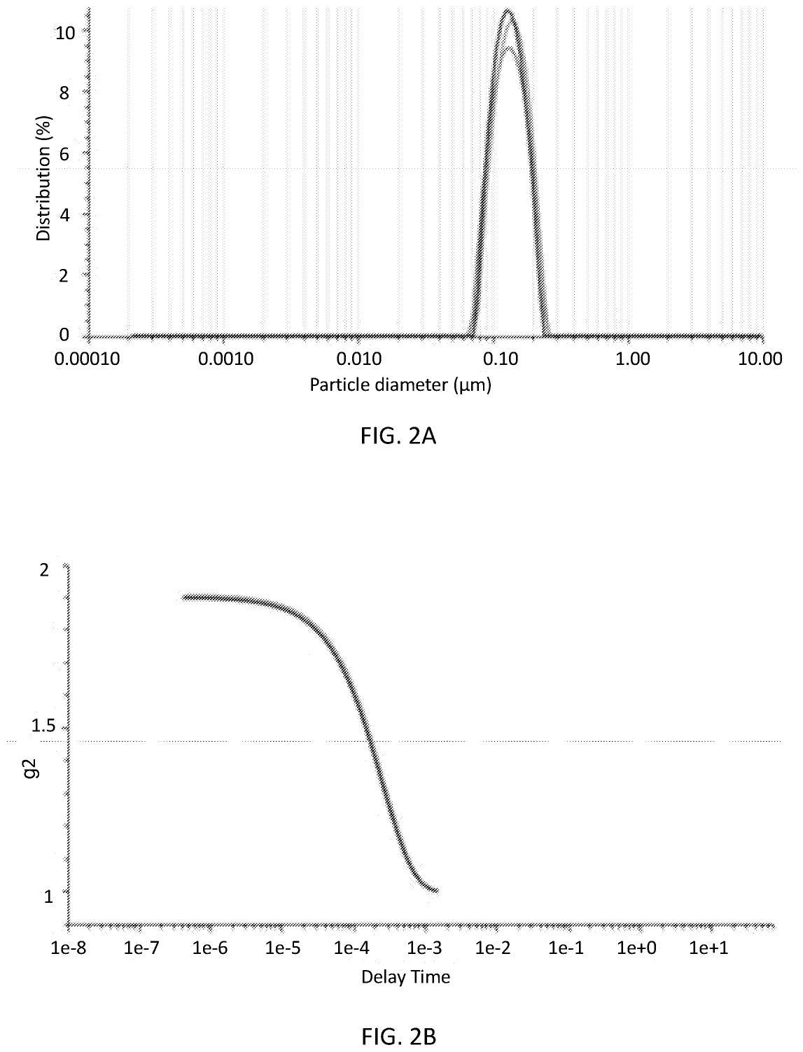

Prior to the use of the DGGR emulsion in a slide layer (e.g., indicator layer), it was analyzed for various Critical to Quality (CTQ) metrics. Dynamic light scattering was used as follows. Emulsions were diluted to 0.1805 g/L using a buffer with 3.75 g/L KNa Tartrate and 0.2 g/L sodium dodecylbenzenesulfonate (SDBS), pH adjusted to 4.0. 1 mL of diluted emulsion was dispensed into a cuvette such that no bubble was visible, and the cuvette was inserted into a DLS instrument (Zetasizer®, Malvern Panalytical). Replicate samples were scanned to assess average particle size and relative polydispersity of the sample. Exemplary results can be seen in FIG. 2A and FIG. 2B, where average particle size was 138 nm, 136 nm and 146 nm in three separate runs, each with a size range of about 70 nm to about 250 nm (FIG. 2A), and the correlation function graph shows confirms the low polydispersity by the steeply-sloped traces and near perfect superposition for the three runs (FIG. 2B). Numerical results are shown in Table 1.

TABLE 1

| | Hydrodynamic | Polydispersity | |
|---|---|---|---|
| Sample | diameter | index | Peak 1 |
| 1 | 0.13767 μm | 18.1% | 0.13773 μm |
| 2 | 0.14574 μm | 12.9% | 0.14236 μm |
| 3 | 0.13642 μm | 16.9% | 0.13502 μm |

### III. Indicator Layer Preparation

2.27 g/L of emulsified DGGR solution was combined with 50 mL of 9.3 mM tartrate buffer (pH 4.0), 3.15 g/L sodium taurodeoxycholate, 9.0% by volume 1-propanol and mixed with an overhead mixer using a square pitch impeller at 250 RPM.

To the DGGR emulsion, 68.0 g/kg of pullulan and 137.0 g/kg of cellulose were added while mixing with an overhead mixer with a square pitch impeller at 250 RPM. Once the pullulan and cellulose were added, the mixing speed was increased to 350 RPM and mixed for 1 hour. Mixing speed was reduced to 250 PRM and 19.5 mL/kg of 10 wt % SDBS solution and 0.5 mL of Proclin 300 per kg were added, followed by mixing for an additional 10 minutes.

The indicator layer was then thoroughly mixed by rolling in a cylindrical container at 15 RPM (about 7.25 cm/sec) for at least 12 hours. The sample was degassed at a vacuum of −28 inches of mercury for 10 minutes prior to adding to a Dynacoater.

In this example, the Dynacoater shim width was 6 inches, with a shim thickness of 0.010 inches. The web gap was 250 μm; the web speed was 2.0 feet/minute; the unwind tension was 5 lbs; and the rewind torque at 20%. The dryer profile was 110×1 and 98×7 (i.e., one dryer was set to 110° F. and seven dryers were set to 98 ºF), with 0.3″ of vacuum. The fluid delivery rate was 11.1 mL/minute resulting in a derived wet thickness of 119 μm and a measured dry thickness of 41.3 μm. The resulting coating layer appeared homogeneous and uniform.

### Example 3—Isolation Layer Preparation and Deposition

To 1.5 mM tartrate buffer (pH 4.0), 2.44 g/kg of Na taurodeoxycholate, 0.5 mL/kg Proclin 300, 0.65 g/kg sodium dodecylbenzenesulfonate (SDBS), and 0.040 g/kg colipase (Roche) were added with stirring using a stir plate and stir bar. 68.0 g/kg of pullulan and 137.0 g/kg of cellulose were added slowly with stirring (200 RPM) under an overhead mixer using square pitch impeller. Once the pullulan and cellulose were added, the mixing speed was increased to 350 RPM and mixed for 1 hour.

The isolation layer was then thoroughly mixed by rolling in a cylindrical container at 15 RPM (about 7.25 cm/sec) for at least 12 hours. The sample was degassed at a vacuum of −28 inches of mercury for 10 minutes prior to adding to a Dynacoater.

In this example, the Dynacoater shim width was 6 inches, with a shim thickness of 0.010 inches. The web gap was 250 μm; the web speed was 2.0 feet/minute; the unwind tension was 5 lbs; and the rewind torque at 20%. The dryer profile was 110×1 and 98×7 (i.e., one dryer was set to 110 ºF and seven dryers were set to 98° F.), with 0.3″ of vacuum. The fluid delivery rate was 12.2 mL/minute resulting in a derived wet thickness of 131 μm. The resulting coating layer appeared homogeneous and uniform.

### Example 4—Basic Buffer Layer Preparation and Deposition

232.0 g/kg of 100 mM bicine buffer solution (adjusted to pH 8.0+/−0.1 with sodium hydroxide pellets or solution) was combined with 342.4 g/kg of 1 wt % carboxymethylcellulose (CMC) solution (10 g/kg sodium carboxymethylcellulose powder; the balance as deionized (DI) water), 3.7 g/kg of polyethylene glycol Mₙ 300 (i.e., PEG-300, or PEG), and DI water to a total volume of 500 mL in a cylindrical container. The basic buffer solution was mixed for 10 minutes with a low (shallow) pitch impeller driven by an overhead mixer at 200 RPM. A 2.25″ 5-blade impeller was used for 500 mL runs.

Particulate matter with a median volume of about 33,500 μm³ was added slowly to the basic buffer mixture to a pigment volume concentration of about 93% with mixing over about 40 minutes) and mixed for an additional 20 minutes at 500 RPM, after which 53.2 g/kg of Hycar Latex HY26652A (Lubrizol) was added and the mixture stirred for 5 minutes at 500 RPM. 5.2 g/kg of sodium taurodeoxycholate was added, followed by 36.8 g/kg of 10 wt % SDBS solution (100 g/kg sodium dodecylbenzenesulfonate powder, balance DI water), followed by stirring 100 RPM for about 2 minutes and then 200 RPM for 5 minutes. The pH of the solution was checked and adjusted as needed to maintain pH 8.0. After each pH adjustment, the mixing container was sealed and rolled in a cylindrical container at about 7.25 cm/sec for at least 20 minutes at before rechecking the pH. The pH-corrected sample was then thoroughly mixed by rolling in a cylindrical container at about 7.25 cm/sec for at least 12 hours. The sample was degassed at a vacuum of −28 inches of mercury for 10 minutes prior to adding to a Dynacoater® (Frontier) and coating onto an optically transparent substrate, in this case Melinex® (DquPont Teijin Films™). Fluid circulation during operation of the coater sufficiently maintained homogeneity of the solution. In this example, the shim width was 6 inches, with a thickness of 0.025 inches. The web gap was 130 μm; the web speed was 2.5 feet/minute; the unwind tension was 5 lbs; and the rewind torque at 20%. The dryer profile was 115×8 (i.e., all eight dryers were set to 115 F), with no vacuum. The fluid delivery rate was 21.8 mL/minute resulting in a derived wet thickness of 188 μm and a measured dry thickness of 151 μm. The resulting coating layer appeared homogeneous and uniform.

Parallel samples were also prepared that were identical to those described above but lacking deoxycholate. The change in viscosity under conditions of increasing shear was measured for samples with and without deoxycholate on the day of sample preparation and the day of sample coating. Samples that included deoxycholate demonstrated more consistent viscosity on the day of preparation versus the day of coating; whereas, samples without deoxycholate demonstrated significant changes in viscosity on the day of sample coating compared to the day of sample preparation.

### Example 5—Stability

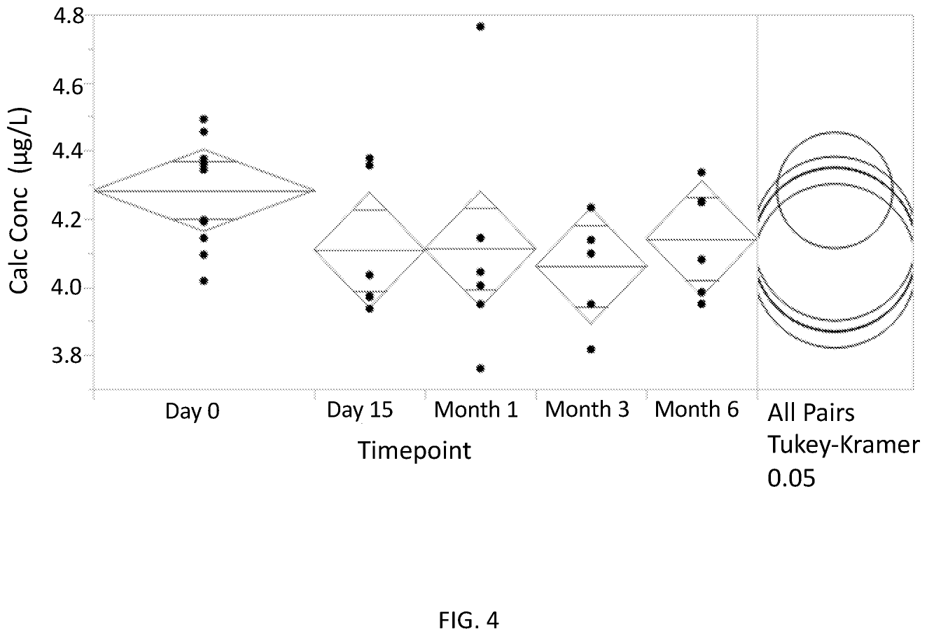

To test the storage stability of dry slides of the description, dry slides stored frozen at −20° C. for 0 days, 15 days, 1 month, 3 months, and 6 months were used to detect feline pancreatic lipase concentration of standardized samples (FIG. 4). No statistically significant changes in calculated concentration were observed over six months of storage.

### Example 6—Sensitivity, Precision

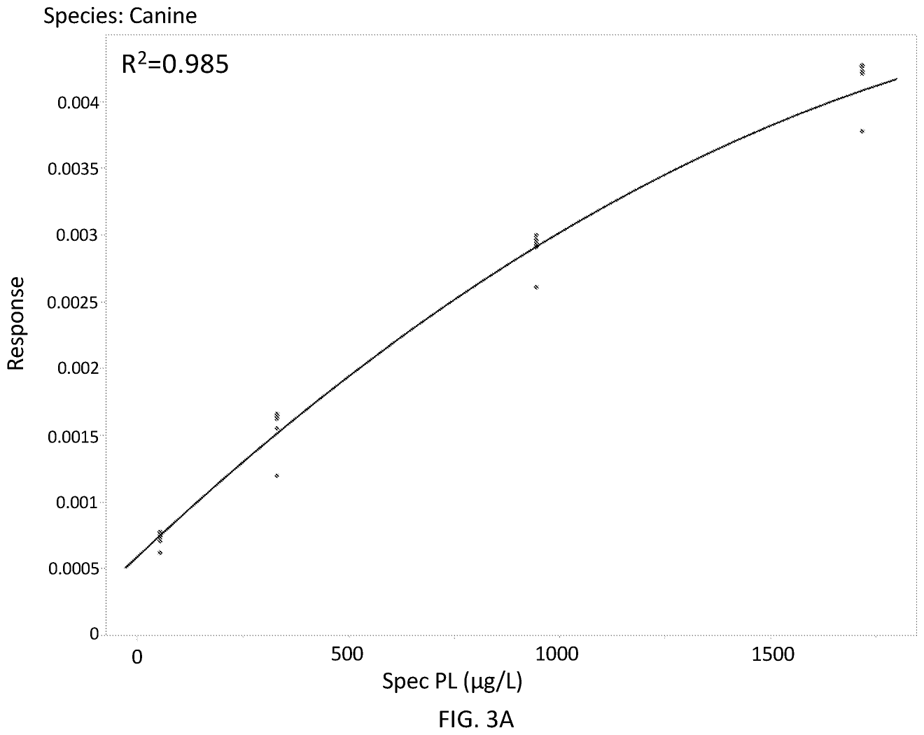

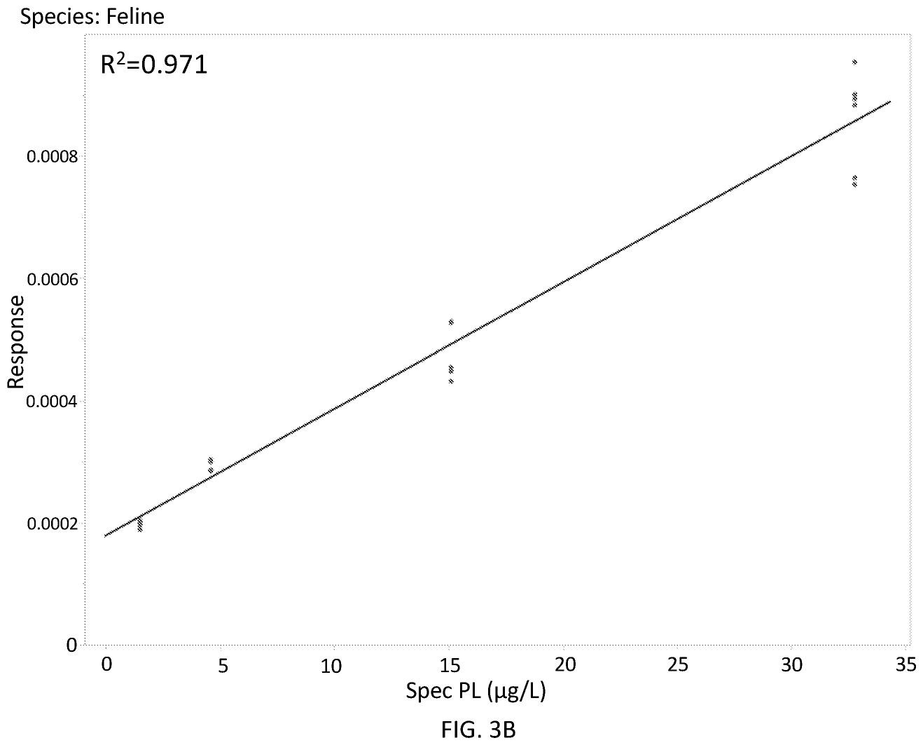

Dose response curves for detection of feline and canine pancreatic lipase were generated, each tested on separately on identical dry slides according to the specification. Both exhibited excellent sensitivity, and precision and also demonstrated strong correlations with a canine-specific test (IDEXX Spec cPL; R²=0.985; FIG. 3A) and a feline-specific test (IDEXX Spec fPL; R²=0.971; FIG. 3B).

Response from the analyzer using the fully-coated dry slide format shows good separation and correlation with Spec cPL/fPL

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims. In addition, the section headings used herein are for organizational purposes only and are not to be construed as limiting the subject matter described.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise.

The present disclosure shall control in the event there are any disparities between any definitions and/or description found in the cited references.

1. A device for detecting the presence or amount of pancreatic lipase in an animal sample comprising:
(a) a basic buffer layer;
(b) an isolation layer comprising colipase; and
(c) an indicator layer comprising a chromogenic substrate.

2. The device of claim 1, further comprising a system for optically determining an amount of color change in a mixture of the sample, the colipase and the chromogenic substrate.

3. (canceled)

4. (canceled)

5. (canceled)

6. The device of claim 1, wherein the chromogenic substrate comprises 1,2-O-dilauryl-rac-glycero-3-glutaric acid-(6-methyl-resorufin) ester (DGGR).

7. (canceled)

8. (canceled)

9. (canceled)

10. The device of claim 1, wherein the basic buffer layer has a pH of between about 7-9.

11. The device of claim 1, wherein the basic buffer layer further comprises one or more surfactants.

12. (canceled)

13. (canceled)

14. (canceled)

15. (canceled)

16. (canceled)

17. The device of claim 1, wherein the basic buffer layer further comprises colipase.

18. (canceled)

19. (canceled)

20. (canceled)

21. The device of claim 1, wherein at least one of the isolation layer and the indicator layer further comprises a support matrix.

22. (canceled)

23. (canceled)

24. (canceled)

25. (canceled)

26. (canceled)

27. (canceled)

28. (canceled)

30. (canceled)

31. The device of claim 21, wherein the isolation layer further comprises a polyvinylpyrrolidone support matrix and a non-ionic surfactant that has a Hydrophilic-Lipophilic Balance (HLB) between about 15-18 and is soluble in an alcohol solvent.

32. The device of claim 1, wherein the isolation layer and the indicator layer each independently further comprises an acidic buffer.

33. The device of claim 32, wherein the acidic buffer has a pH of between about 3.0 to 5.0.

34. (canceled)

35. The device of claim 1, wherein at least one of the isolation layer and the indicator layer each independently further comprises one or more bile salts.

36. (canceled)

37. (canceled)

38. (canceled)

39. The device of claim 1, wherein at least one of the isolation layer and the indicator layer each independently further comprises one or more surfactants.

40. (canceled)

41. (canceled)

42. (canceled)

43. (canceled)

44. The device of claim 1, further comprising a primer layer beneath the indicator layer.

45. (canceled)

46. (canceled)

47. The device of claim 1, further comprising an optically transparent substrate beneath the indicator layer.

48. (canceled)

49. (canceled)

50. (canceled)

51. (canceled)

52. (canceled)

53. The device of claim 1, wherein the basic buffer layer further comprises one or more bile salts and calcium.

54. (canceled)

55. (canceled)

56. (canceled)

57. (canceled)

58. (canceled)

59. A method for detecting the presence or amount of pancreatic lipase in an animal sample comprising:
i. providing a device comprising:
(a) a basic buffer layer;
(b) an isolation layer comprising colipase;
(c) an indicator layer comprising a chromogenic substrate; and
(d) a system for optically determining the amount of a development of a color change in the mixture of the sample, the colipase and the chromogenic substrate;
ii. introducing the sample onto the basic buffer layer for diffusion through the basic buffer layer, the isolation layer, and the indicator layer; and
iii. determining a color change in the device, and
iv. correlating the color change to the presence or amount of pancreatic lipase in the sample.

60. The method of claim 59, wherein the animal is a canine or a feline.

61. The method of claim 59, wherein the animal sample comprises whole blood, serum or plasma.

62. The method of claim 61, wherein the animal sample comprises serum.

63. The method of claim 59, wherein the chromogenic substrate comprises 1,2-O-dilauryl-rac-glycero-3-glutaric acid-(6-methyl-rsorufin) ester (DGGR).

64. The method of claim 63, wherein the DGGR is emulsified.

65. (canceled)

66. (canceled)

67. The method of claim 59, wherein the basic buffer layer has a pH of between about 7-9.

68. (canceled)

69. (canceled)

70. (canceled)

71. (canceled)

72. (canceled)

73. (canceled)

74. The method of claim 59, wherein the basic buffer layer further comprises colipase.

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. The method of claim 59, wherein at least one of the isolation layer and the indicator layer further comprises an acidic buffer.

89. The method of claim 88, wherein the acidic buffer has a pH of between about 3.0 to 5.0.

90. (canceled)

91. The method of claim 59, wherein at least one of the isolation layer and the indicator layer further comprises one or more bile salts.

92. (canceled)

93. (canceled)

94. (canceled)

95. The method of claim 59, wherein at least one of the isolation layer and the indicator layer further comprises one or more surfactants.

96. (canceled)

97. (canceled)

98. (canceled)

99. (canceled)

100. The method of claim 59, further comprising a primer layer beneath the indicator layer.

101. (canceled)

102. (canceled)

103. The method of claim 59, further comprising an optically transparent substrate beneath the indicator layer.

104. (canceled)

105. (canceled)

106. (canceled)

107. (canceled)

108. (canceled)

109. (canceled)

110. (canceled)

111. (canceled)

112. (canceled)

113. (canceled)

114. A kit comprising, the device of claim 1, and instructions for use of the device.
